(19) United States
(12) Patent Application Publication
(10) Pub. No.: US 2025/0161522 A1
(43) Pub. Date: May 22, 2025

(54) AIR STERILIZER AND AIR PURIFYING APPARATUS COMPRISING SAME

(71) Applicant: Samsung Electronics Co., Ltd., Suwon-si (KR)

(72) Inventors: Huiyoung KIM, Suwon-si (KR); Sunghyun CHUN, Suwon-si (KR); Nakhyun KIM, Suwon-si (KR); Juyoung KIM, Suwon-si (KR); Juwan PARK, Suwon-si (KR); Changhyun PARK, Suwon-si (KR); Changwoo JUNG, Suwon-si (KR); Seoyoung CHO, Suwon-si (KR)

(21) Appl. No.: 19/034,107

(22) Filed: Jan. 22, 2025

Related U.S. Application Data
(63) Continuation of application No. PCT/KR2023/013224, filed on Sep. 5, 2023.

(30) Foreign Application Priority Data
Oct. 6, 2022 (KR) 10-2022-0128141
Nov. 2, 2022 (KR) 10-2022-0144730

(51) Int. Cl. A61L 9/20

(52) U.S. Cl. CPC A61L 9/20; A61L 2209/14

(57) ABSTRACT

An air sterilizer is provided. The air sterilizer includes a case module having a length extending in a first direction, and a main body module of which at least a portion is arranged inside the case module and which comprises an ultraviolet sterilizer module generating ultraviolet rays, wherein the case module includes a case forming at least a portion of an exterior and having a hollow structure providing a space for accommodating the ultraviolet sterilizer module therein, and a case fixing structure inserted and assembled into the case in the first direction and configured to be fixed to the main body module, wherein one of the case and the case fixing structure is provided with a hook and the other one of the case and the case fixing structure is provided with a hook fixing groove into which the hook is inserted and which fixes a position of the hook in the first direction, such that the case and the case fixing structure are fixed in position in the first direction by assembly.

### CROSS-REFERENCE TO RELATED APPLICATION(S)

This application is a continuation application, claiming priority under § 365(c), of an International application No. PCT/KR2023/013224, filed on Sep. 5, 2023, which is based on and claims the benefit of a Korean patent application number 10-2022-0128141, filed on Oct. 6, 2022, in the Korean Intellectual Property Office, and of a Korean patent application number 10-2022-0144730, filed on Nov. 2, 2022, in the Korean Intellectual Property Office, the disclosure of each of which is incorporated by reference herein in its entirety.

### BACKGROUND

### 1. Field

The disclosure relates to an air sterilizer and an air cleaning device including the same.

### 2. Description of Related Art

An air cleaning device may be a device for improving the quality of indoor air in various indoor spaces such as homes or offices and may include, for example, an air cleaner that removes foreign substances such as fine dust by using a filter.

Additionally, the air cleaning device may include, for example, an air sterilizer configured to sterilize various bacteria or viruses in indoor air to improve the quality of the indoor air. The air sterilizer may include a case and an ultraviolet lamp that is arranged in the case and generates ultraviolet rays to remove bacteria and the like in air that has flowed into the case.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide an air sterilizer and an air cleaning device including the same.

Additional aspects will be set forth in part in the description which follows and, in part, will be apparent from the description, or may be learned by practice of the presented embodiments.

In accordance with an aspect of the disclosure, an air sterilizer is provided. The air sterilizer includes a case module having a length extending in a first direction, and a main body module of which at least a portion is arranged inside the case module and which includes an ultraviolet sterilizer module generating ultraviolet rays, wherein case module includes a case forming at least a portion of an exterior and having a hollow structure providing a space for accommodating the ultraviolet sterilizer module therein, and a case fixing structure inserted and assembled into the case in the first direction and configured to be fixed to the main body module, and wherein one of the case and the case fixing structure is provided with a hook and the other one of the case and the case fixing structure is provided with a hook fixing groove in which the hook is inserted and which fixes a position of the hook in the first direction, such that the case and the case fixing structure are fixed in position in the first direction by assembly.

In accordance with another aspect of the disclosure, an air cleaning device is provided. The air cleaning device includes an air cleaner configured to remove dust in air, and the air sterilizer configured to sterilize the air from which dust has been removed, wherein the air sterilizer includes a case module having a length extending in a first direction, and a main body module arranged inside the case module and including an ultraviolet sterilizer module generating ultraviolet rays, wherein the case module includes a case forming at least a portion of an exterior and having a hollow structure providing a space for accommodating the ultraviolet sterilizer module therein; and a case fixing structure inserted and assembled into the case in the first direction and configured to be fixed to the main body module, and wherein one of the case and the case fixing structure is provided with a hook and the other one of the case and the case fixing structure is provided with a hook fixing groove into which the hook is inserted and which fixes a position of the hook in the first direction, such that the case and the case fixing structure are fixed in position in the first direction by assembly.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view illustrating an air sterilizer according to an embodiment of the disclosure;

FIG. 2 is an exploded perspective view illustrating an air sterilizer according to an embodiment of the disclosure;

FIG. 3 is an exploded perspective view illustrating a main body module of an air sterilizer according to an embodiment of the disclosure;

FIG. 4 is a cross-sectional view of an air sterilizer according to an embodiment of the disclosure;

FIG. 5 is a cross-sectional view for describing a state in which a case module is fixed to a main body module in an air sterilizer according to an embodiment of the disclosure;

FIG. 6 is an assembled perspective view illustrating a state in which a case and a case fixing structure of a case module are assembled according to an embodiment of the disclosure;

FIG. 7 is an exploded perspective view illustrating a state in which a case and a case fixing structure are separated from each other in a case module according to an embodiment of the disclosure;

FIG. 8 is a cross-sectional view for describing a process in which a case fixing structure and a case are assembled in a case module according to an embodiment of the disclosure;

FIG. 9 is a cross-sectional view illustrating a state in which a case fixing structure and a case are assembled in a case module according to an embodiment of the disclosure;

FIG. 10 is an enlarged view of a portion of FIG. 9 according to an embodiment of the disclosure;

FIG. 11 is a perspective view illustrating an example of a case according to an embodiment of the disclosure;

FIG. 12A is a perspective view illustrating an example of a case according to an embodiment of the disclosure, and FIG. 12B is a cross-sectional view illustrating a state in which a case fixing structure is assembled to a case of FIG. 12A according to an embodiment of the disclosure;

FIG. 13 is a cross-sectional view illustrating an assembled state of a case and a case fixing structure in a case module according to an embodiment of the disclosure;

FIG. 14 is a perspective view for describing an anti-rotation unit of a case module according to an embodiment of the disclosure;

FIG. 15 is a diagram for describing operation of an anti-rotation unit of FIG. 14 according to an embodiment of the disclosure;

FIG. 16 is a diagram for describing examples of a cross-sectional shape of a case according to an embodiment of the disclosure;

FIG. 17 is a perspective view illustrating an air cleaning device according to an embodiment of the disclosure; and

FIG. 18 is a cross-sectional view of an air cleaning device of FIG. 17 according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Although terms such as “first” and “second” may be used herein to describe various elements, the elements should not be limited by the terms. These terms are only used to distinguish one element from another element. For example, without departing from the scope of the disclosure, a first element may be referred to as a second element, and vice versa. As used herein, the term “and/or” includes any one or any combination of the associated listed items.

The terms used herein are used to describe an embodiment and are not intended to restrict and/or limit the disclosure. It will be understood that terms such as “comprise”, “include”, and “have”, when used herein, specify the presence of stated features, integers, steps, operations, elements, components, or combinations thereof, but do not preclude the presence or addition of one or more other features, integers, steps, operations, elements, components, or combinations thereof. In the drawings, like reference symbols denote like members that perform substantially the same function.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

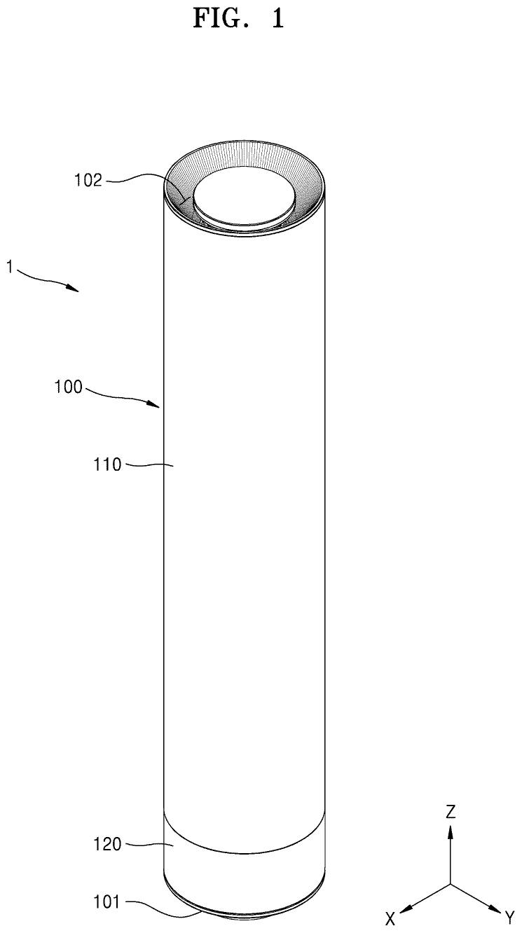

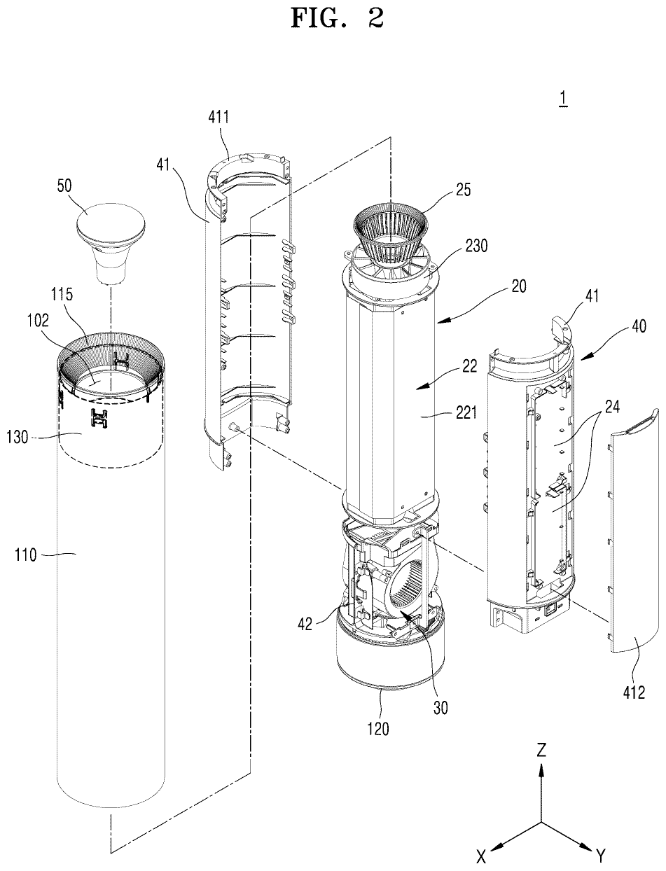

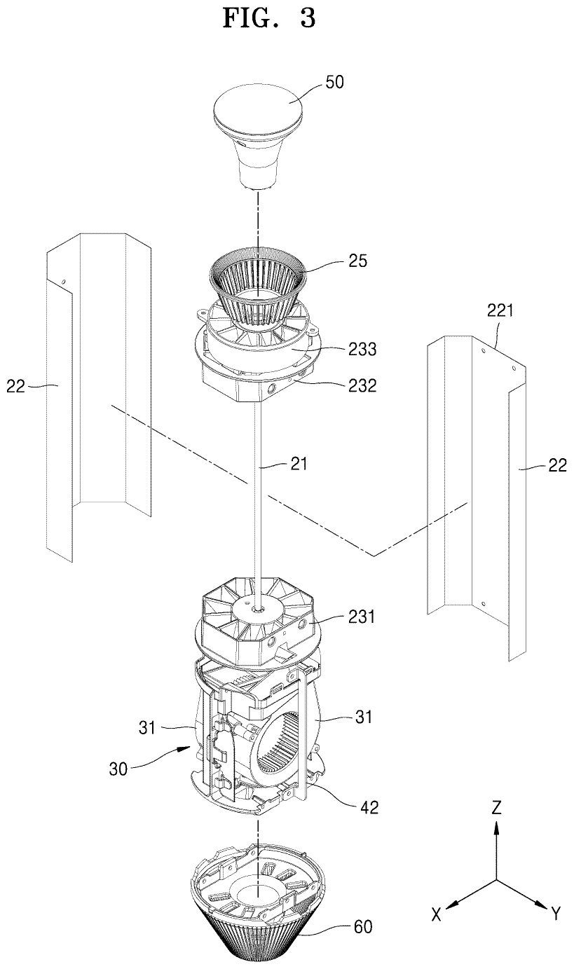

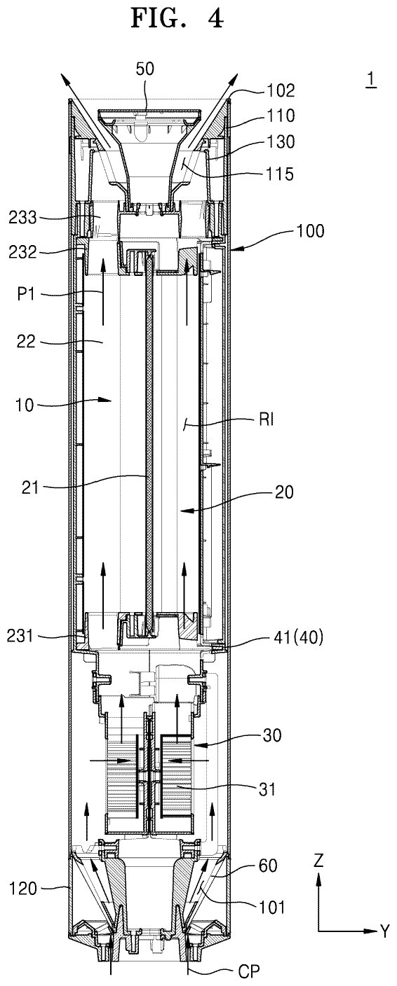

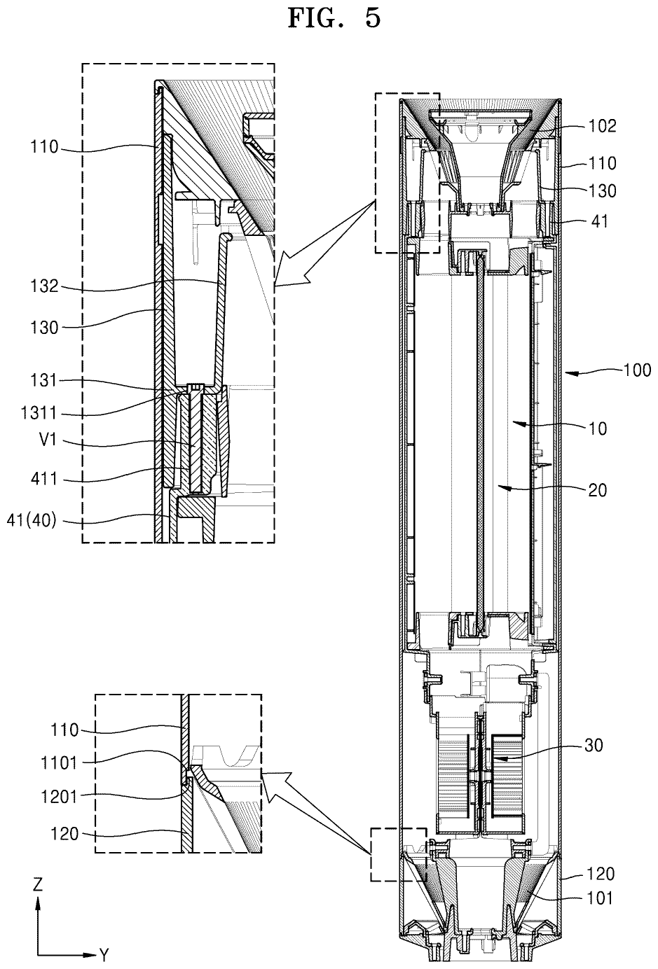

FIG. 1 is a perspective view illustrating an air sterilizer 1 according to an embodiment of the disclosure, and FIG. 2 is an exploded perspective view illustrating an air sterilizer 1 according to an embodiment of the disclosure. FIG. 3 is an exploded perspective view illustrating a main body module 10 of an air sterilizer 1 according to an embodiment of the disclosure. FIG. 4 is a cross-sectional view of an air sterilizer 1 according to an embodiment of the disclosure, and FIG. 5 is a cross-sectional view for describing a state in which a case module 100 is fixed to a main body module 10 in an air sterilizer 1 according to an embodiment of the disclosure.

Referring to FIGS. 1 to 4, the air sterilizer 1 according to an embodiment may be configured to sterilize air introduced from the outside and discharge the sterilized air back to the outside. The air sterilizer 1 may include an inlet 101 and an outlet 102, and a path P1 through which air moves from the inlet 101 toward the outlet 102 may be formed therein.

The air sterilizer 1 may include a case module 100 forming an exterior, and a main body module 10 of which at least a portion is arranged inside the case module 100.

In an embodiment, the main body module 10 may include an ultraviolet sterilizer module 20 generating ultraviolet rays, and a blower module 30 configured to form an air flow toward the ultraviolet sterilizer module 20.

The ultraviolet sterilizer module 20 may be arranged on the path P1. The ultraviolet sterilizer module 20 may include at least one ultraviolet lamp 21 arranged on the path P1. The ultraviolet lamp 21 may generate ultraviolet rays and may be singular or plural.

The ultraviolet lamp 21 may be arranged to irradiate ultraviolet rays to the air introduced through the inlet 101. The ultraviolet lamp 21 may generate ultraviolet C (UVC) to remove bacteria in the air. The ultraviolet lamp 21 may generate light with a wavelength of 220 nm to 280 nm. The ultraviolet lamp 21 may remove contaminants such as bacteria and viruses in the introduced air by irradiating ultraviolet rays to the introduced air.

In another embodiment, the ultraviolet sterilizer module 20 may include a reflection cover 22 arranged to reflect the ultraviolet rays irradiated by the ultraviolet lamp 21. The reflection cover 22 may cover the outside of the ultraviolet lamp 21. The reflection cover 22 may be arranged in the path P1.

At least a portion of the reflection cover 22 may include a metal material such that an inner surface facing the ultraviolet lamp 21 reflects ultraviolet rays. As an example, the inner surface of the reflection cover 22 may include a material with high light reflectivity. For example, the inner surface of the reflection cover 22 may be plated with a metal such as chrome. For another example, all of the reflection cover 22, including the inner surface thereof, may include a plate of a metal material with high light reflectivity.

The reflection cover 22 may have a bent shape. For example, the reflection cover 22 may include a plurality of flat plates, and a plurality of plate shapes thereof may be arranged to form a certain angle therebetween and surround the ultraviolet lamp 21.

The reflection cover 22 may have a polygonal cross-sectional shape. For example, the cross-sectional shape of the reflection cover 22 may be designed in consideration of the arrangement of a printed circuit board assembly 24 described below. For another example, the cross-sectional shape of the reflection cover 22 may have an asymmetrical shape in which a plate 221 facing the printed circuit board assembly 24 is relatively larger than other plates.

The reflection cover 22 may be provided as a plurality of reflection covers 22. For example, each of the plurality of reflection covers 22 may cover the outer side of the ultraviolet lamp 21. For example, some of the plurality of reflection covers 22 may cover one side of the ultraviolet lamp 21, and some other of the plurality of reflection covers 22 may cover the other side of the ultraviolet lamp 21. The shape and number of the reflection covers 22 are not limited thereto and may be variously modified to reflect ultraviolet rays irradiated by the ultraviolet lamp 21. For example, the reflection cover 22 may be provided in singularity. The reflection cover 22 may be manufactured by bending a single plate.

The ultraviolet sterilizer module 20 may further include a light blocking member 230 arranged to prevent the ultraviolet rays irradiated by the ultraviolet lamp 21 from being incident to the outside of an irradiation region RI. The light blocking member 230 may be arranged in the path P1. The light blocking member 230 may be formed to allow the air flowing along the path P1 to pass therethrough. The light blocking member 230 may be arranged to allow air to flow along the path P1 while preventing the ultraviolet rays irradiated by the ultraviolet lamp 21 from being emitted to the outside of the irradiation region RI.

The light blocking member 230 may be arranged upstream and downstream of the ultraviolet lamp 21 along the path P1. For example, the light blocking member 230 may include a first light blocking member 231, a second light blocking member 232, and a third light blocking member 233. The first light blocking member 231 may be arranged upstream of the ultraviolet lamp 21, and the second light blocking member 232 and the third light blocking member 233 may be arranged downstream of the ultraviolet lamp 21. Through the light blocking member 230, the components of an air cleaner 200 arranged outside the ultraviolet sterilizer module 20 may be prevented from being damaged by the ultraviolet rays, and the ultraviolet rays may be prevented from being emitted to the outside of the air sterilizer 1 through the outlet 102.

In an embodiment, the first light blocking member 231 and the second light blocking member 232 may be arranged to support the ultraviolet lamp 21. For example, the first light blocking member 231 and the second light blocking member 232 may be arranged to support both ends of the ultraviolet lamp 21.

The blower module 30 may generate a flow inside the air sterilizer 1 and may be arranged in the path P1. For example, the blower module 30 may be configured such that the air introduced into the air sterilizer forms an air flow toward the ultraviolet sterilizer module 20.

The ultraviolet sterilizer module 20 and the blower module 30 may be arranged along the path P1. For example, the ultraviolet sterilizer module 20 and the blower module 30 may be arranged in a first direction Z. For example, the blower module 30 may be arranged under the ultraviolet sterilizer module 20. The blower module 30 may be arranged between the inlet 101 and the ultraviolet sterilizer module 20. When the blower module 30 is driven, the air introduced through the inlet 101 may sequentially pass through the blower module 30 and the ultraviolet sterilizer module 20 and then be discharged through the outlet 102. The arrangement of the blower module 30 is not necessarily limited thereto, and the blower module 30 may be arranged over the ultraviolet sterilizer module 20.

The blower module 30 may include a plurality of blowers 31. The plurality of blowers 31 may be centrifugal blowers. The plurality of blowers 31 may have shapes corresponding to each other. The plurality of blowers 31 may be symmetrically arranged around a central axis of the case module 100.

Each of the plurality of blowers 31 may be arranged to discharge air toward the ultraviolet sterilizer module 20. A direction inclined with respect to the first direction corresponding to the direction in which air is discharged by some of the plurality of blowers 31 and a direction inclined with respect to the first direction corresponding to the direction in which air is discharged by some other of the plurality of blowers 31 may be opposite to each other.

The shape, arrangement, number, and the like of the blowers 31 of the blower module 30 are not limited thereto and may be variously modified. For example, the blower 31 may be another type of blower 31 such as an axial-flow blower, or the blower 31 may be provided in singularity.

The main body module 10 may further include an inner frame 40. The inner frame 40 may be arranged inside the case module 100. The inner frame 40 may be arranged to support at least some of the components of the air sterilizer 1.

For example, the inner frame 40 may include a first support frame 41 supporting the ultraviolet sterilizer module 20. The first support frame 41 may support the printed circuit board assembly 24 and a control panel 50. The first support frame 41 may include a board mounting cover 412 covering the printed circuit board assembly 24.

The inner frame 40 may further include a second support frame 42 supporting the blower module 30. The second support frame 42 may be coupled to the first support frame 41. The configuration of the inner frame 40 is not limited thereto, and the first support frame 41 and the second support frame 42 may be configured as a single body or the first support frame 41 or the second support frame 42 may be omitted.

The case module 100 may form at least a portion of the exterior of the air sterilizer 1, and the ultraviolet sterilizer module 20 and the blower module 30 may be arranged therein. For example the case module 100 may include the inlet 101 for introducing air and the outlet 102 for discharging air.

The case module 100 may be divided into a first case 110 and a lower case 120. The outlet 102 may be arranged at the first case 110, and the inlet 101 may be arranged at the lower case 120. For example, the outlet 102 may be arranged at an end portion, for example, an upper end, of the first case 110, and the inlet 101 may be arranged at an end portion, for example, a lower end, of the lower case 120. An upper cover 115 with the outlet 102 may be arranged over the first case 110.

The control panel 50 may be arranged at an end portion of the first case 110 so as to overlap the outlet 102 in the first direction Z. The control panel 50 may be arranged apart from the outlet 102 by a certain distance such that air is discharged through the outlet 102. In an embodiment, the control panel 50 may be supported on the inner frame 40 by an outlet grill 25.

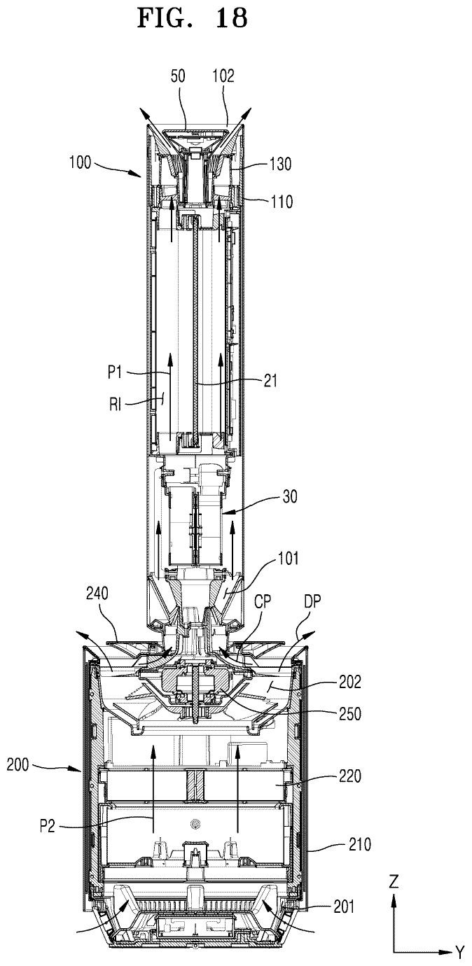

A base unit 60 for supporting the blower module 30 may be arranged inside the lower case 120. The blower module 30 and the second support frame 42 may be arranged on the base unit 60. The base unit 60 may be configured for connection for an air flow to an air cleaner 200 (see FIG. 18) described below. For example, by the base unit 60, at least a portion of the air discharged through a second outlet 202 of the air cleaner 200 may be introduced through the inlet 101 of the air sterilizer 1.

The shape of the case module 100 and the arrangement of the outlet 102 and the inlet 101 are not limited thereto and may be variously modified. For example, the case module 100 may have different ratios of the first case 110 and the lower case 120 or may include only the first case 110 without the lower case 120. For example, the arrangement of the outlet 102 and the inlet 101 is also not limited thereto and may be variously modified as long as it may provide air around the ultraviolet lamp 21 and discharge the sterilized air.

The first case 110 (hereinafter referred to as ‘case 110’) may form at least a portion of the exterior of the air sterilizer 1 and may have a hollow structure providing a space for accommodating the ultraviolet sterilizer module 20. The case 110 may have a cylindrical structure. The ultraviolet sterilizer module 20 and the blower module 30 may be arranged inside the case 110.

The case 110 may have a length extending in the first direction Z, for example, in the up/down direction. For example, the case 110 may have a shape in which the length in the first direction Z is greater than the width in a second direction Y that is perpendicular to the first direction Z. For another example, the length of the case 110 in the first direction Z may be three times to seven times the width of the case 110 in the second direction Y.

The case 110 may include a single body. For example, the case 110 may have a structure without any seam in the circumferential direction. For example, the case 110 may have a cylindrical shape and may have a structure without any seam in the circumferential direction. In another embodiment, the case 110 may improve the aesthetic sense of the air sterilizer 1 by providing a continuous surface without any seam in the circumferential direction.

In addition, the main body module 10 of the air sterilizer 1 may have a longer lifetime or may be semipermanent without a replacement cycle, compared to the internal components of the air cleaner 200 described below. For example, the lifetime of the ultraviolet sterilizer module 20 of the air sterilizer 1 may be equal to the lifetime of the air sterilizer 1. Accordingly, even when the case 110 is configured as a single body in order to improve the aesthetic sense, the user's inconvenience may not be great.

In order to form a seamless case 110, an extrusion process may be used to manufacture the case 110. By the extrusion process, the case 110 may have a pipe shape having a hollow structure. For example, the cross-sectional shape of the case 110 in a direction perpendicular to the first direction Z may be circular.

The case 110 may include metal. In an example, the material of the case 110 may include at least one of aluminum, steel, zinc, or an alloy thereof. By including the metal, the case 110 may provide a unique aesthetic sense and tactile feel of the metal.

Referring to FIGS. 4 and 5, the case module 100 may be configured such that the case 110 is fixed to the main body module 10. For example, the case module 100 may be configured such that the case 110 is fixed to the inner frame 40 of the main body module 10.

A portion of the case 110 may be supported by the lower case 120. For example, one end portion of the case 110 may be supported by an end portion of the lower case 120. A seating groove 1201 may be arranged at an upper end of the lower case 120, and a seating protrusion 1101 inserted into the seating groove 1201 may be arranged at a lower end of the case 110. The diameter of the case 110 may be equal to the diameter of the lower case 120. The lower end of the case 110 may be arranged at a certain position by arranging the case 110 on the lower case 120 such that the seating protrusion 1101 of the case 110 is held in the seating groove 1201 of the lower case 120.

Another portion of the case 110 may be fixed to the main body module 10. For example, the case module 100 may include a case fixing structure 130 configured to fix the case 110 to the main body module 10.

In an example, the case fixing structure 130 may be fixed to the inner surface of the case 110, and by fixing the case fixing structure 130 to the main body module 10, the case 110 may be fixed to the main body module 10.

Referring to FIGS. 2 and 5, the case fixing structure 130 may include a main body fixing unit 131 fixed to the main body module 10. The main body fixing unit 131 may be provided with a fastening hole 1311 for fixing a fastening member V1. The fastening member V1 may be inserted into the fastening hole 1311 in the first direction Z and may be fastened to the inner frame 40 of the main body module 10 through the fastening hole 1311 and thus the case fixing structure 130 may be fixed to the main body module 10. The first support frame 41 of the inner frame 40 may include a second fastening hole 411 for coupling the fastening member V1 thereto.

In the case module 100 according to an embodiment, because the case 110 is fixed to the main body module 10 by the case fixing structure 130 arranged inside the case 110, the fastening member V1 for fixing the case 110 to the main body module 10 may not be exposed to the outside of the case 110.

Additionally, in the case module 100 according to an embodiment, a structure in which an assembly structure of the case 110 and the case fixing structure 130 is not exposed to the outside of the case 110 may be provided.

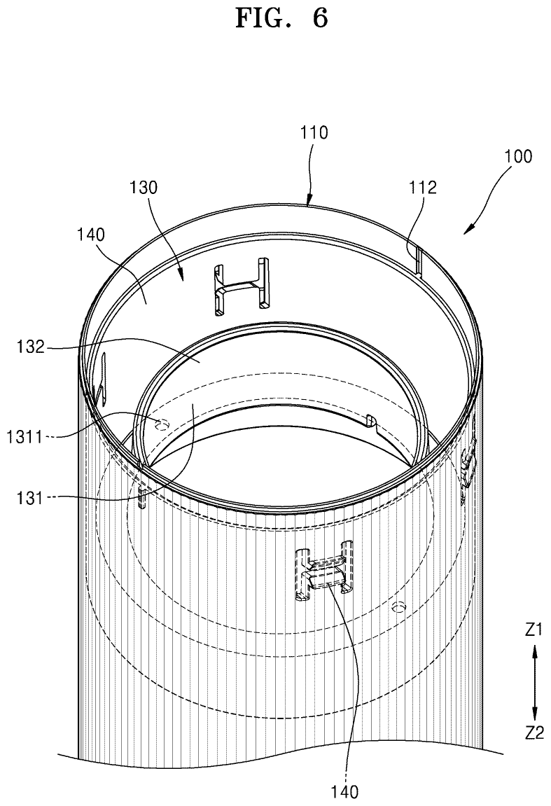

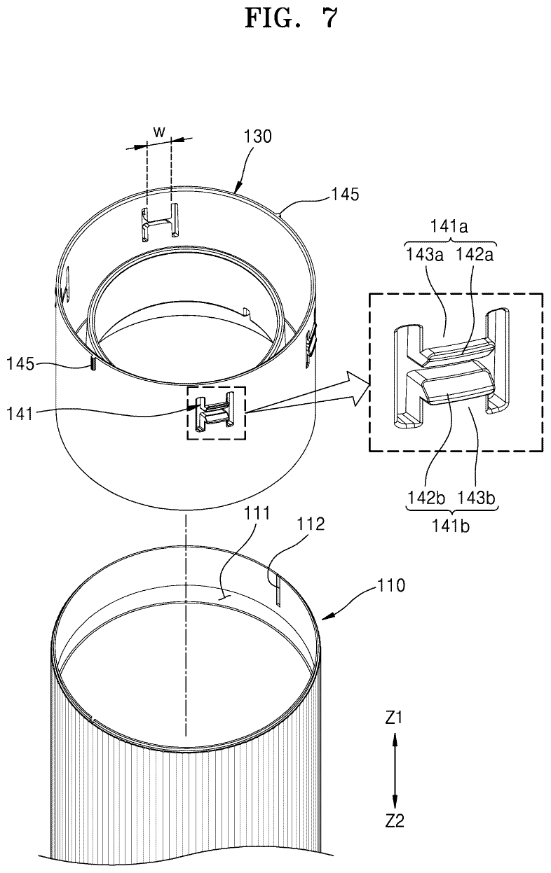

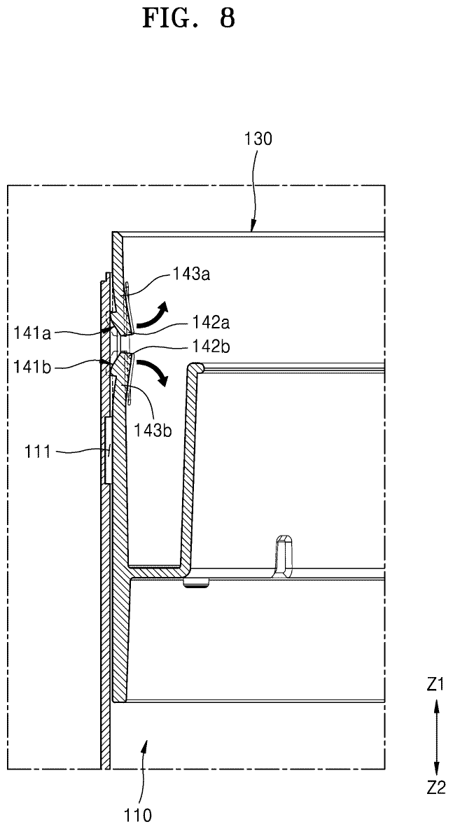

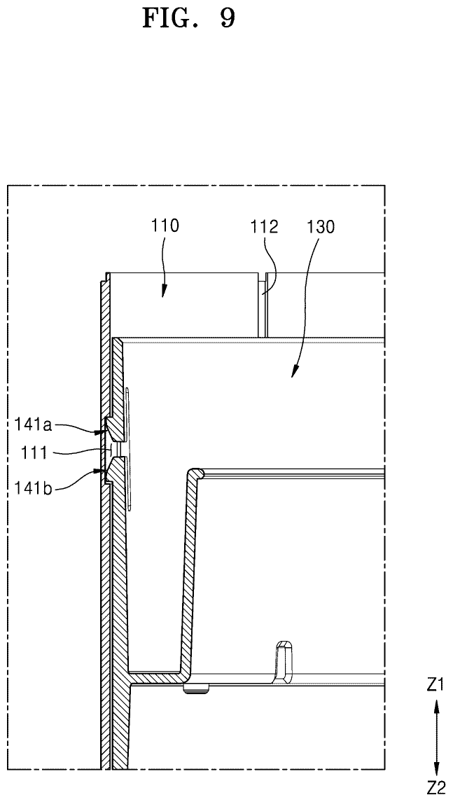

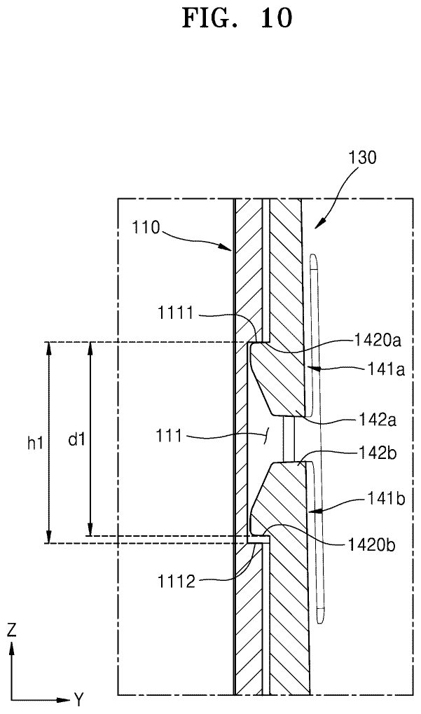

FIG. 6 is an assembled perspective view illustrating a state in which a case 110 and a case fixing structure 130 of a case module 100 are assembled according to an embodiment of the disclosure, and FIG. 7 is an exploded perspective view illustrating a state in which a case 110 and a case fixing structure 130 are separated from each other in a case module 100 according to an embodiment of the disclosure. FIG. 8 is a cross-sectional view for describing a process in which a case fixing structure 130 and a case 110 are assembled in a case module 100 according to an embodiment of the disclosure. FIG. 9 is a cross-sectional view illustrating a state in which a case fixing structure 130 and a case 110 are assembled in a case module 100 according to an embodiment of the disclosure. FIG. 10 is an enlarged view of a portion of FIG. 9 according to an embodiment of the disclosure.

Referring to FIGS. 6 to 9, the case fixing structure 130 may be inserted and assembled into the case 110 in the first direction. The case fixing structure 130 may be configured to be inserted and assembled in one direction among the first directions, for example, in the upward direction, or to be inserted and assembled in another direction among the first directions, for example, in the downward direction.

The case fixing structure 130 may be manufactured separately from the case 110 and assembled to the case 110. The case fixing structure 130 may further include a case fixing unit 140 that is arranged to be assembled to the case 110. The case fixing unit 140 may be arranged at the edge of the main body fixing unit 131. The case fixing structure 130 may further include a grill support unit 132 for supporting the outlet grill 25. In the case fixing structure 130, the case fixing unit 140, the main body fixing unit 131, and the grill support unit 132 may be configured as a single body.

The case fixing structure 130 may be arranged inside the case 110, and accordingly, the material selection and process selection may be relatively free compared to the case 110.

The case fixing structure 130 may include a different material than the case 110. In an example, the case fixing structure 130 may include resin. The case fixing structure 130 may be manufactured by an injection process. The case fixing structure 130 may have a relatively complex structure compared to the case 110.

The case fixing structure 130 may be assembled to the case 110, and thus, the case 110 and the case fixing structure 130 may be fixed in position with respect to each other in the first direction Z. In order for the case 110 and the case fixing structure 130 to be fixed in position in the first direction Z by assembly, one of the case 110 and the case fixing structure 130 may be provided with a hook 141 and the other one of the case 110 and the case fixing structure 130 may be provided with a hook fixing groove 111 into which the hook 141 is inserted and which fixes the position of the hook 141 in the first direction Z. For example, the hook 141 may be elastically deformable in a direction intersecting the first direction Z.

When the materials of the case 110 and the case fixing structure 130 are different from each other, the hook 141 and the hook fixing groove 111 may be arranged in consideration of the materials. For example, when the case 110 includes metal and the case fixing structure 130 includes resin, the hook fixing groove 111 with a relatively simple structure among the hook 141 and the hook fixing groove 111 may be arranged at the case 110, and the hook 141 with a relatively complex structure may be arranged at the case fixing structure 130. The hook 141 may be arranged on the outer surface of the case fixing structure 130, and the hook fixing groove 111 may be arranged on the inner surface of the case 110. The arrangement of the hook 141 and the hook fixing groove 111 is not necessarily limited thereto and may be variously modified in consideration of the materials and processing methods of the case 110 and the case fixing structure 130.

The case fixing structure 130 may include the hook 141 that is elastically deformable in a direction intersecting the first direction Z. The inner surface of the case 110 may be provided with the hook fixing groove 111 into which the hook 141 is inserted and which fixes the position of the hook 141 in the first direction Z.

In an embodiment, the hook fixing groove 111 may have a groove shape extending along the inner surface of the case 110. Because the hook fixing groove 111 has a groove shape, it may not be exposed to the outside of the case 110. The hook fixing groove 111 may extend in the circumferential direction.

The width of the hook fixing groove 111 extending in the circumferential direction may be greater than a width w of the hook 141. When the width of the hook fixing groove 111 is designed to exactly match the width w of the hook 141, the width of the hook 141 may become greater than a designed width or the position of the hook 141 may be modified due to the manufacturing tolerance in the manufacturing process. Accordingly, an assembly defect may occur, such as the hook 141 not being inserted into the hook fixing groove 111 or the hook 141 being damaged. On the contrary, in the case module 100 according to an embodiment, the width of the hook fixing groove 111 may be formed to be greater than the width w of the hook 141, thereby preventing the occurrence of an assembly defect between the hook 141 and the hook fixing groove 111.

As an example in which the width of the hook fixing groove 111 is greater than the width w of the hook 141, the hook fixing groove 111 may have a closed-loop structure that extends continuously in the circumferential direction. In other words, the hook fixing groove 111 may have a ring shape. However, the shape of the hook fixing groove 111 is not necessarily limited thereto.

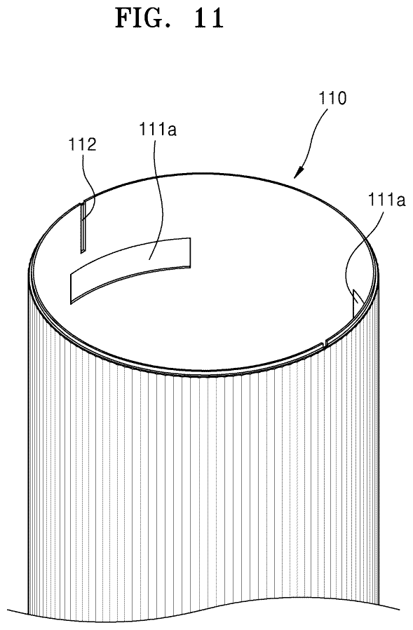

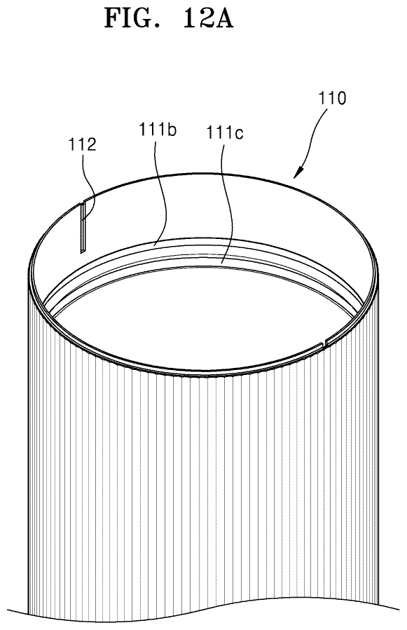

FIG. 11 is a perspective view illustrating an example of a case according to an embodiment of the disclosure.

Referring to FIG. 11, a plurality of hook fixing grooves 111a may be arranged apart from each other in the circumferential direction along the inner surface.

Referring back to FIG. 7, the hook 141 may be arranged at the case fixing structure 130 and may have a structure in which at least a portion thereof protrudes toward the outer surface. The hook 141 may include a protrusion portion (142a, 142b) and a banding portion (143a, 143b) that may be bent such that the protrusion portion (142a, 142b) may be movable in a direction intersecting the first direction Z.

The hook 141 may include a first hook unit 141a for fixing the position of the case 110 in one direction among the first directions Z with respect to the case fixing structure 130, and a second hook unit 141b for fixing the position of the case 110 in the first direction Z with respect to the case fixing structure 130. For example, the first hook unit 141a may be inserted into the hook fixing groove 111 to block the case 110 from moving in one direction among the first directions Z, for example, the downward direction Z2. In another example, the second hook unit 141b may be inserted into the hook fixing groove 111 to block the case 110 from moving in another direction among the first directions Z, for example, the upward direction Z1.

The first hook unit 141a may include a first protrusion portion 142a and a first band portion 143a that may be bent such that the first protrusion portion 142a may be movable in a direction intersecting the first direction Z. In an embodiment, the second hook unit 141b may include a second protrusion portion 142b and a second band portion 143b that may be bent such that the second protrusion portion 142b may be movable in a direction intersecting the first direction Z.

Referring to FIGS. 8 and 9, in the process of inserting and assembling the case fixing structure 130 into the case 110, the hook 141 may be bent in a direction intersecting the first direction Z by contacting the inner surface of the case 110. As the case fixing structure 130 is further inserted in the first direction Z, for example, the downward direction Z2, the hook 141 may be inserted into the hook fixing groove 111 and the protrusion portion (142a, 142b) of the hook 141 may be restored to its original position.

As illustrated in FIG. 8, in the process of inserting the case fixing structure 130 in the downward direction, until the hook 141 is inserted into the hook fixing groove 111, the first protrusion portion 142a of the first hook unit 141a and the second protrusion portion 142b of the second hook unit 141b may be pressed by the inner surface of the case 110 and thus retreat in a direction intersecting the first direction Z, for example, to the right side. In this case, the first band portion 143a and the second band portion 143b may be bent toward the inside of the case fixing structure 130.

In this state, when the case fixing structure 130 is further inserted in the downward direction Z2 and thus the hook 141 reaches the hook fixing groove 111 as illustrated in FIG. 9, the first protrusion portion 142a of the first hook unit 141a and the second protrusion portion 142b of the second hook unit 141b may be released from the pressure by the inner surface of the case 110 and thus be restored to their original positions. Accordingly, the first protrusion portion 142a and the second protrusion portion 142b may be inserted into the hook fixing groove 111.

Referring to FIGS. 9 and 10, the first protrusion portion 142a of the first hook unit 141a and the second protrusion portion 142b of the second hook unit 141b may be inserted into the hook fixing groove 111, a first lock surface 1420a of the first protrusion portion 142a may face a first step 1111 of the hook fixing groove 111, and a second lock surface 1420b of the second protrusion portion 142b may face a second step 1112 of the hook fixing groove 111.

In another embodiment, the first lock surface 1420a and the first step 1111 may have surfaces that are perpendicular to the first direction Z, and the second lock surface 1420b and the second step 1112 may have surfaces that are perpendicular to the first direction Z. By the first lock surface 1420a of the first protrusion portion 142a and the first step 1111 of the hook fixing groove 111, the case 110 may be restricted from moving in the downward direction Z2 with respect to the case fixing structure 130. By the second lock surface 1420b of the second protrusion portion 142b and the second step 1112 of the hook fixing groove 111, the case 110 may be restricted from moving in the upward direction Z1 with respect to the case fixing structure 130. The movement in the first direction Z between the case 110 and the case fixing structure 130 in both directions may be restricted by the hook 141 including the first hook unit 141a and the second hook unit 141b.

The first hook unit 141a and the second hook unit 141b may be arranged to face each other in the first direction Z. A distance dl between the first protrusion portion 142a and the second protrusion portion 142b in the first direction Z may correspond to a height h1 of the hook fixing groove 111 in the first direction Z. For example, the distance dl between the first protrusion portion 142a and the second protrusion portion 142b in the first direction Z may be equal to or slightly less than the height h1 of the hook fixing groove 111 in the first direction Z. The difference between the distance d1 between the first protrusion portion 142a and the second protrusion portion 142b and the height h1 of the hook fixing groove 111 may be 0.5 mm or less.

The distance d1 between the first protrusion portion 142a and the second protrusion portion 142b in the first direction Z may be the distance between the first lock surface 1420a and the second lock surface 1420b. The height h1 of the hook fixing groove 111 in the first direction Z may be the distance between the first step 1111 and the second step 1112 of the hook fixing groove 111. The distance between the first lock surface 1420a and the second lock surface 1420b may correspond to the distance between the first step 1111 and the second step 1112 of the hook fixing groove 111. The distance between the first lock surface 1420a and the second lock surface 1420b may be equal to or slightly less than the distance between the first step 1111 and the second step 1112 of the hook fixing groove 111. For example, the difference between the distance between the first lock surface 1420a and the second lock surface 1420b and the distance between the first step 1111 and the second step 1112 of the hook fixing groove 111 may be 2 mm or less.

The number of hook fixing grooves 111 may be less than the number of hooks 141. For example, when the hook 141 includes the first hook unit 141a and the second hook unit 141b in the first direction Z, the hook fixing groove 111 may be singular in the first direction Z. However, the number of hook fixing grooves 111 and hooks 141 is not necessarily limited thereto and may be variously modified.

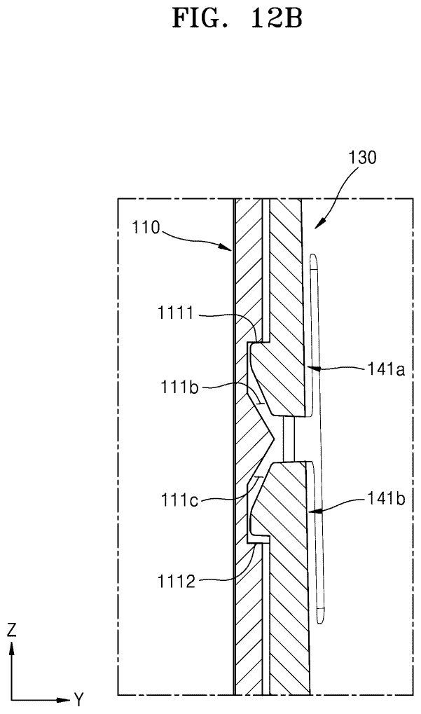

FIG. 12A is a perspective view illustrating an example of a case according to an embodiment of the disclosure, and FIG. 12B is a cross-sectional view illustrating a state in which a case fixing structure is assembled to the case of FIG. 12A according to an embodiment of the disclosure.

Referring to FIGS. 12A and 12B, a hook fixing groove (111b, 111c) may include a first hook fixing groove 111b into which a first hook unit 141a is insertable, and a second hook fixing groove 111c into which a second hook unit 141b is insertable. In this case, a first step 1111 may be arranged at an upper end of the first hook fixing groove 111b, and a second step 1112 may be arranged at a lower end of the second hook fixing groove 111c.

As another example, the hook 141 may be singular in the first direction Z.

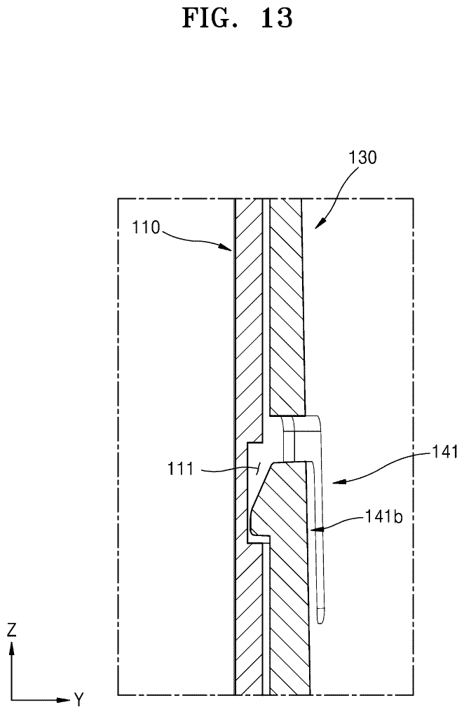

FIG. 13 is a cross-sectional view illustrating an assembled state of a case and a case fixing structure in a case module according to an embodiment of the disclosure.

Referring to FIG. 13, the hook 141 may include one hook unit 141b for fixing the position of the case 110 in one direction among the first directions Z with respect to the case fixing structure 130.

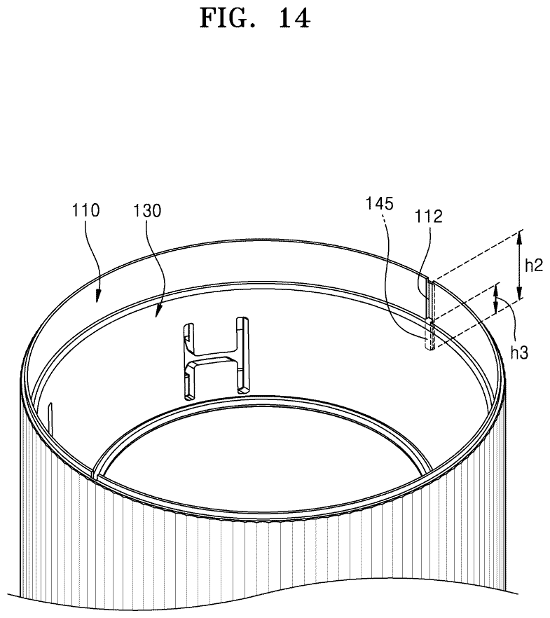

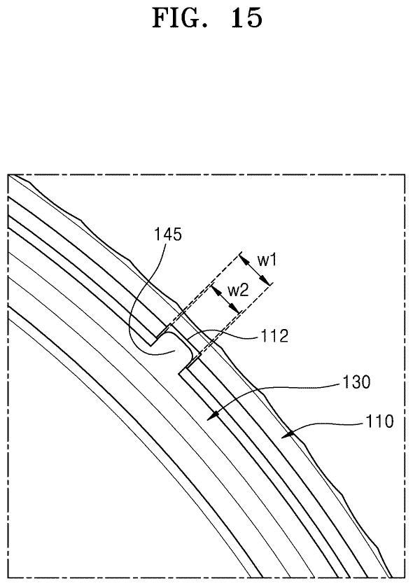

FIG. 14 is a perspective view for describing an anti-rotation unit of a case module 100 according to an embodiment of the disclosure, and FIG. 15 is a diagram for describing operation of the anti-rotation unit of FIG. 14 according to an embodiment of the disclosure.

Referring to FIGS. 7, 14, and 15, the case module 100 may further include an anti-rotation unit configured to prevent the case 110 from rotating with respect to the case fixing structure 130.

The anti-rotation unit may include an anti-rotation protrusion 145 provided on one of the case 110 and the case fixing structure 130, and an anti-rotation slit 112 provided in the other one of the case 110 and the case fixing structure 130. The anti-rotation protrusion 145 may be inserted into the anti-rotation slit 112, and the rotation thereof may be blocked by the anti-rotation slit 112.

For example, at least one anti-rotation slit 112 may be provided in the inner surface of the case 110, and at least one anti-rotation protrusion 145 may be provided on the outer surface of the case fixing structure 130. For example, a plurality of anti-rotation slits 112 may be provided in the inner surface of the case 110, and a plurality of anti-rotation protrusions 145 may be provided on the outer surface of the case fixing structure 130. As an example, the number of anti-rotation slits 112 may be equal to the number of anti-rotation protrusions 145. For another example, the number of anti-rotation slits 112 may be less than the number of anti-rotation protrusions 145.

The anti-rotation slit 112 may extend in the insertion direction of the case fixing structure 130, for example, a direction parallel to the first direction Z. The anti-rotation slit 112 may have a height h2 in the first direction Z. The anti-rotation slit 112 may have a width w1 in the circumferential direction perpendicular to the first direction Z. The anti-rotation slit 112 may have a groove shape so as not to be exposed to the outer surface of the case 110.

In an embodiment, the anti-rotation protrusion 145 may be inserted into the anti-rotation slit 112 in the first direction Z. A height h3 of the anti-rotation protrusion 145 in the first direction Z may be less than the height h2 of the anti-rotation slit 112 in the first direction Z. When the anti-rotation protrusion 145 is inserted into the anti-rotation slit 112, the movement in the circumferential direction perpendicular to the first direction Z may be restricted. As an example thereof, a width w2 of the anti-rotation protrusion 145 in the circumferential direction may correspond to the width w1 of the anti-rotation slit 112 in the circumferential direction. The width w2 of the anti-rotation protrusion 145 in the circumferential direction may be equal to the width w1 of the anti-rotation slit 112 in the circumferential direction. Even in consideration of the manufacturing tolerance, the difference between the width w2 of the anti-rotation protrusion 145 and the width w1 of the anti-rotation slit 112 may be less than 0.3 mm.

In an embodiment, an example in which the anti-rotation protrusion 145 and the anti-rotation slit 112 of the anti-rotation unit are plural and are arranged at the case fixing structure 130 and the case 110 respectively has been mainly described; however, the disclosure is not necessarily limited thereto. For example, the anti-rotation protrusion 145 and the anti-rotation slit 112 of the anti-rotation unit may be singular. For another example, even when the anti-rotation protrusion 145 and the anti-rotation slit 112 of the anti-rotation unit are plural, the anti-rotation protrusion 145 and the anti-rotation slit 112 may be arranged in the inner surface of the case 110 and another anti-rotation slit 112 and another anti-rotation protrusion 145 corresponding to the anti-rotation slit 112 and the anti-rotation protrusion 145 arranged at the case 110 may be arranged on the outer surface of the case fixing structure 130.

As described above, by including the anti-rotation unit, the case module 100 may prevent the case 110 from rotating with respect to the case fixing structure 130, while securing a stable coupling between the hook 141 and the hook fixing groove 111.

The configuration of the anti-rotation unit may be omitted depending on the cross-sectional shape of the case 110. For example, when the cross-sectional shape of the case 110 in the first direction is not circular, the anti-rotation unit may be omitted.

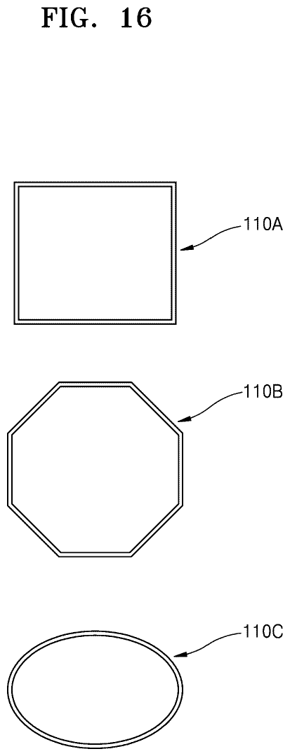

FIG. 16 is a diagram for describing examples of a cross-sectional shape of a case according to an embodiment of the disclosure.

Referring to FIG. 16, when the cross-sectional shape of a case (110A, 110B, 110C) in the first direction is elliptical or polygonal such as tetragonal or octagonal, the case module 100 may not include the anti-rotation unit.

In the case module 100 according to the above configuration, the case 110 may be assembled and fixed to the case fixing structure 130 by the hook 141 and the hook fixing groove 111 without any externally-exposed fastening member. Thus, because a portion for fixing the case 110 to the main body module 10 is not exposed to the outside of the air sterilizer 1, the aesthetic sense of the product may be further improved.

In addition, by separating the fastening member V1 fixing the case fixing structure 130 and the main body module 10 to each other, the case 110 may be easily separated from the main body module 10.

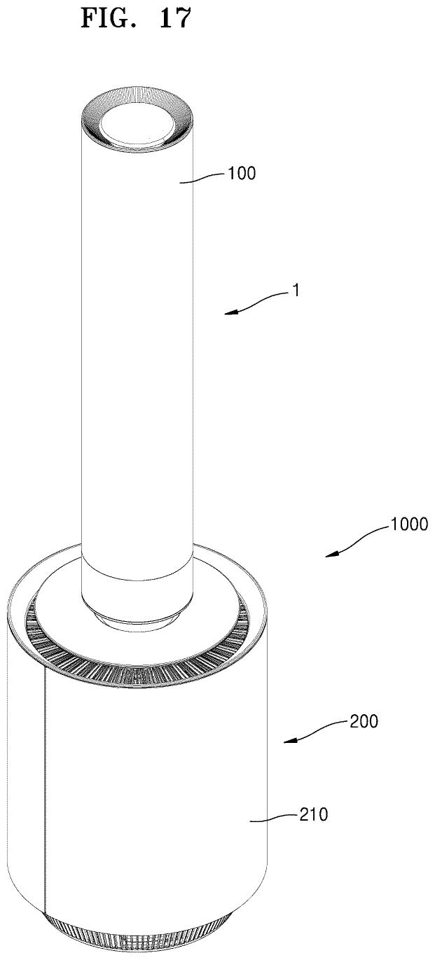

FIG. 17 is a perspective view illustrating an air cleaning device 1000 according to an embodiment of the disclosure, and FIG. 18 is a cross-sectional view of the air cleaning device 1000 of FIG. 17 according to an embodiment of the disclosure.

Referring to FIGS. 17 and 18, the air cleaning device 1000 may be a device arranged to control the properties of air such as temperature, humidity, cleanliness, and air flow. For example, the air cleaning device 1000 may be a device for increasing the cleanliness of indoor air.

The air cleaning device 1000 according to the embodiment may include an air cleaner 200 and the air sterilizer 1 according to the embodiment described above.

The air cleaner 200 may be configured to purify the air introduced from the outside and discharge the purified air to the outside of the air cleaner 200. In an example, the air cleaner 200 may include a second inlet 201 and a second outlet 202. The air cleaner 200 may include a second path P2 arranged between the second inlet 201 and the second outlet 202.

The air cleaner 200 may include a second case 210, a dust collection filter 220 arranged inside the second case 210, and a second blower module 250. The dust collection filter 220 and the second blower module 250 may be arranged in the second path P2. The air cleaner 200 may, for example, include a discharge path guide 240. The discharge path guide 240 may be arranged to guide an air flow on a discharge path DP.

The air sterilizer 1 may be assembled to be connected to the air cleaner 200. The air sterilizer 1 may be detachably assembled to the air cleaner 200. The inlet 101 of the air sterilizer 1 may be connected to the air cleaner 200, and accordingly, the air from which dust has been removed by the air cleaner 200 may be sterilized by the ultraviolet sterilizer module 20.

A connection path CP may be arranged between the second outlet 202 of the air cleaner 200 and the inlet 101 of the air sterilizer 1. When the blower module 30 of the air sterilizer 1 and the second blower module 250 of the air cleaner 200 are simultaneously driven, the discharge path guide 240 may guide at least a portion of the air discharged through the second outlet 202 (other than a portion introduced into the path P1 through the connection path CP) to flow along the discharge path DP. For example, the discharge path guide 240 may cover at least a portion of the second outlet 202 from the outside.

In the embodiment of FIGS. 17 and 18, an example in which the air sterilizer 1 is used as a component of the air cleaning device 1000 has been mainly described; however, the use of the air sterilizer 1 according to an embodiment is not limited thereto. For example, the air sterilizer 1 may be used as a component of a device other than the air cleaning device 1000 or may be independently used as a separate device from another device.

In the above embodiment, an example in which the assembly structure of the main body module 10 and the case module 100 is applied to the air sterilizer 1 has been mainly described; however, the disclosure is not necessarily limited thereto and may be applied to various devices. For example, the assembly structure of the case 110 and the case fixing structure 130 of the case module 100 may be applied to the air cleaner 200. In another example, the assembly structure of the case 110 and the case fixing structure 130 described above may be applied to an electronic device including a main body module and a case module accommodating the main body module.

For the sake of understanding of the disclosure, reference symbols are used in embodiments illustrated in the drawings and particular terms are used to describe the embodiments; however, the disclosure is not limited by the particular terms and the disclosure may include all components that may be generally thought by those of ordinary skill in the art.

Particular implementations described herein are merely embodiments, and do not limit the scope of the disclosure in any way. For the sake of conciseness, descriptions of related art electronic configurations, control systems, software, and other functional aspects of the systems may be omitted. The connections or connection members of lines between components illustrated in the drawings illustrate functional connections and/or physical or circuit connections, and in actual devices, they may be represented as various replaceable or additional functional connections, physical connections, or circuit connections. When there is no particular statement such as “essential” and “important”, it may not be a necessary component for the application of the disclosure. Expressions such as “comprising” and “including” used herein are to be understood as open-ended terms of the description.

The use of the terms “a”, “an”, and “the” and similar indicative terms in the specification of the disclosure (particularly in the attached claims) may be construed to cover both the singular and the plural. Also, when a range is stated herein, it may include the disclosure to which individual values within the range are applied (unless there is a statement to the contrary) and it may be the same as the statement of each individual value constituting the range. Moreover, the operations of a method according to the disclosure may be performed in any suitable order unless otherwise stated herein or otherwise clearly contradicted by the context. The scope of the disclosure is not limited to the stated order of the operations. All examples or example terms (e.g., “such as”) used herein are merely intended to describe the disclosure in detail, and the scope of the disclosure is not limited by the examples or example terms unless otherwise defined in the attached claims. Also, those of ordinary skill in the art may clearly understand that various modifications and changes may be easily made therein without departing from the scope and spirit of the disclosure.

The air sterilizer according to an embodiment may provide an aesthetic sense of the product through a case having a smooth exterior without any seam in the circumferential direction.

An air sterilizer may include a case module having a length extending in a first direction, and a main body module of which at least a portion is arranged inside the case module and which includes an ultraviolet sterilizer module generating ultraviolet rays, wherein the case module may include a case forming at least a portion of an exterior and having a hollow structure providing a space for accommodating the ultraviolet sterilizer module therein, and a case fixing structure inserted and assembled into the case in the first direction and configured to be fixed to the main body module.

In the case module, one of the case and the case fixing structure may be provided with a hook and the other one of the case and the case fixing structure may be provided with a hook fixing groove in which the hook is inserted and which fixes a position of the hook in the first direction, such that the case and the case fixing structure are fixed in position in the first direction by assembly.

The hook fixing groove may be provided in an inner surface of the case, and the hook may be provided on an outer surface of the case fixing structure.

The case may, for example, have a circular cross-sectional shape in a direction perpendicular to the first direction, and the case module may further include an anti-rotation unit configured to prevent the case from rotating with respect to the case fixing structure.

The anti-rotation unit may include an anti-rotation protrusion provided on one of the case and the case fixing structure, and an anti-rotation slit provided in the other one of the case and the case fixing structure, into which the anti-rotation protrusion is inserted, and which blocks rotation of the anti-rotation protrusion.

The hook fixing groove may extend along an inner surface of the case in a circumferential direction, and an extension width of the hook fixing groove may be greater than a width of the hook.

The hook fixing groove may, for example, have a closed loop structure extending continuously along an inner surface of the case.

The hook may include a first hook unit for fixing a position of the case in one direction among the first direction with respect to the case fixing structure, and a second hook unit for fixing a position of the case in another direction opposite to the one direction among the first direction with respect to the case fixing structure.

The first hook unit and the second hook unit may be arranged to face each other in the first direction.

Materials of the case and the case fixing structure may be different.

The case may, for example, include metal, and the case fixing structure may include resin.

The case fixing structure may include a main body fixing unit fixed to the main body module, wherein the main body fixing unit may be provided with a plurality of fastening holes for fixing a plurality of fastening members.

The case module may be provided with an inlet at a lower portion thereof and an outlet at an upper portion thereof.

According to one embodiment, an air cleaning device may include an air cleaner configured to remove dust in air, and an air sterilizer configured to sterilize the air from which dust has been removed, wherein the air sterilizer may include a case module having a length extending in a first direction, and a main body module arranged inside the case module and including an ultraviolet sterilizer module generating ultraviolet rays, wherein the case module may include a case forming at least a portion of an exterior and having a hollow structure providing a space for accommodating the ultraviolet sterilizer module therein, and a case fixing structure inserted and assembled into the case in the first direction and configured to be fixed to the main body module.

In the case module, one of the case and the case fixing structure may be provided with a hook and the other one of the case and the case fixing structure may be provided with a hook fixing groove into which the hook is inserted and which fixes a position of the hook in the first direction, such that the case and the case fixing structure are fixed in position in the first direction by assembly.

In an embodiment, the case may have a circular cross-sectional shape in a direction perpendicular to the first direction, and the case module may further include an anti-rotation unit configured to prevent the case from rotating with respect to the case fixing structure.

The hook fixing groove may be provided in an inner surface of the case, the hook may be provided on an outer surface of the case fixing structure, and a width of the hook fixing groove may be greater than a width of the hook.

The air sterilizer according to an embodiment may provide an aesthetic sense of the product through a case having a smooth exterior without any seam in the circumferential direction.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An air sterilizer comprising:
a case module having a length extending in a first direction; and
a main body module of which at least a portion is arranged inside the case module and which comprises an ultraviolet sterilizer module generating ultraviolet rays,
wherein the case module comprises:
a case forming at least a portion of an exterior and having a hollow structure providing a space for accommodating the ultraviolet sterilizer module therein, and
a case fixing structure inserted and assembled into the case in the first direction and configured to be fixed to the main body module, and
wherein one of the case and the case fixing structure is provided with a hook and the other one of the case and the case fixing structure is provided with a hook fixing groove into which the hook is inserted and which fixes a position of the hook in the first direction, such that the case and the case fixing structure are fixed in position in the first direction by assembly.

2. The air sterilizer of claim 1,
wherein the hook fixing groove is provided in an inner surface of the case, and
wherein the hook is provided on an outer surface of the case fixing structure.

3. The air sterilizer of claim 1,
wherein the case has a circular cross-sectional shape in a direction perpendicular to the first direction, and
wherein the case module further comprises an anti-rotation unit configured to prevent the case from rotating with respect to the case fixing structure.

4. The air sterilizer of claim 3, wherein the anti-rotation unit comprises:
an anti-rotation protrusion provided on one of the case and the case fixing structure; and
an anti-rotation slit provided in the other one of the case and the case fixing structure, into which the anti-rotation protrusion is inserted, and which blocks rotation of the anti-rotation protrusion.

5. The air sterilizer of claim 1,
wherein the hook fixing groove extends along an inner surface of the case in a circumferential direction, and
wherein an extension width of the hook fixing groove is greater than a width of the hook.

6. The air sterilizer of claim 1, wherein the hook fixing groove has a closed loop structure extending continuously along an inner surface of the case.

7. The air sterilizer of claim 1, wherein the hook comprises:
a first hook unit for fixing a position of the case in one direction among the first direction with respect to the case fixing structure; and
a second hook unit for fixing a position of the case in another direction opposite to the one direction among the first direction with respect to the case fixing structure.

8. The air sterilizer of claim 7, wherein the first hook unit and the second hook unit are arranged to face each other in the first direction.

9. The air sterilizer of claim 1, wherein materials of the case and the case fixing structure are different.

10. The air sterilizer of claim 1,
wherein the case comprises metal, and
wherein the case fixing structure comprises resin.

11. The air sterilizer of claim 1,
wherein the case fixing structure comprises a main body fixing unit fixed to the main body module, and
wherein the main body fixing unit is provided with a plurality of fastening holes for fixing a plurality of fastening members.

12. The air sterilizer of claim 1, wherein the case module is provided with an inlet at a lower portion thereof and an outlet at an upper portion thereof.

13. An air cleaning device comprising:
an air cleaner configured to remove dust in air; and
an air sterilizer configured to sterilize the air from which dust has been removed,
wherein the air sterilizer comprises:
a case module having a length extending in a first direction, and a main body module arranged inside the case module and comprising an ultraviolet sterilizer module generating ultraviolet rays,
wherein the case module comprises:
a case forming at least a portion of an exterior and having a hollow structure providing a space for accommodating the ultraviolet sterilizer module therein; and a case fixing structure inserted and assembled into the case in the first direction and configured to be fixed to the main body module, and
wherein one of the case and the case fixing structure is provided with a hook and the other one of the case and the case fixing structure is provided with a hook fixing groove into which the hook is inserted and which fixes a position of the hook in the first direction, such that the case and the case fixing structure are fixed in position in the first direction by assembly.

14. The air cleaning device of claim 13,
wherein the case has a circular cross-sectional shape in a direction perpendicular to the first direction, and
wherein the case module further comprises an anti-rotation unit configured to prevent the case from rotating with respect to the case fixing structure.

15. The air cleaning device of claim 13,
wherein the hook fixing groove is provided in an inner surface of the case,
wherein the hook is provided on an outer surface of the case fixing structure, and
wherein a width of the hook fixing groove is greater than a width of the hook.
